(19) United States
(12) Patent Application Publication
(10) Pub. No.: US 2001/0055779 A1
(43) Pub. Date: Dec. 27, 2001

(54) Competition assay for detecting compound-protein interactions in situ

(76) Inventor: Glenna C. Burmer, Seattle, WA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/816,040

(22) Filed: Mar. 21, 2001

Related U.S. Application Data
(60) Provisional application No. 60/191,362, filed on Mar. 22, 2000.

(51) Int. Cl. G01N 33/567

(52) U.S. Cl. 435/7.21

(57) ABSTRACT

The described invention includes methods and assays to identify compounds that interact with a binding domain of a protein by a competition assay with a labeled ligand targeted to the peptides of the binding domain of that protein. These assays can be used for high throughput compound screening, or used to identify sites of compound interaction with proteins in tissue samples.

### CROSS-REFERENCES TO RELATED APPLICATIONS

The present application claims priority to U.S. Ser. No. 60/191,362, filed Mar. 22, 2000, herein incorporated by reference in its entirety.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

High throughput compound screening methods in drug discovery traditionally involve analyzing thousands of compounds against expressed proteins in solution, or cell lines in vitro within a 96 or 384 microtiter assay plate, or tissue samples on a membrane. Recent advances in high density array and microchip technology reduce the volume of these interactions as well as increase the number of substances that can be assayed by increasing the density of the bound substances in a microarray format. If the objective is to detect binding, however, the compound must still be tagged or a conformational change or change in activity must be detected in the protein during the binding assay, which is not conducive to the high density array format technology. Also, it is fortuitous to conduct a binding assay on the selected tissue or cells directly which is not feasible with the high density array to date.

The current invention describes general methods for detecting binding between a compound with a protein by using a competition assay between the compound being screened and ligands targeted to an active site or ligand binding domain of the protein. These methods are generally applicable to screening any compound-protein interaction, and can be used both in vitro and in situ to detect proteins in tissue sections and cultured cells.

### SUMMARY OF THE INVENTION

The described invention includes methods and assays to identify compounds that interact with an active site or binding domain of a target protein by a competition assay with a ligand targeted to the active site or binding domain of the target protein. These assays can be used for high throughput compound screening, and can also be used to identify sites of compound interaction with target proteins in in situ tissue samples or cultured cells.

The detection of compound-target protein interactions has many potential uses in biological research and drug discovery. The methods and assays described here can aid in drug discovery. New compounds can be identified and tested for targeting a protein of interest. The assays can enable the identification of potential or actual localized sites of interaction between a compound and a tissue or diseased tissue or organ of interest. Thus, the assays can be used to confirm that a target organ will bind the compound of interest. Further, potential sites of unexpected interaction between a drug and an organ can be identified. These assays can identify unexpected side effects due to binding between the drug and a tissue that is an unexpected site of drug interaction. Other applications will be readily ascertained by those of skill in the art.

### DETAILED DESCRIPTION OF THE INVENTION

Introduction

The subject invention disclosed herein is a method for detecting binding between a compound and a target protein by using a competition assay between the compound being screened and ligands such as antibodies targeted, e.g., to the active site or binding domain of the protein, which compete for binding with the compound for binding to the target. This method is particularly useful for in vitro and in situ applications with cultured cells and tissue samples. The assays described here can be used to identify new compounds that bind to a target protein of interest or to identify the active site or binding domain on a target protein of interest. The assay can also be used to determine whether or not a drug or other compound of interest binds a tissue of interest.

In the method, a ligand is obtained that is specific for a target protein of interest. The ligand is known to bind the protein or target of interest. A “ligand” is a molecule that is recognized by a particular receptor or which otherwise specifically binds a protein, e.g., at an active site or binding domain. Typically the nature of the interaction or binding is non-covalent, e.g., by hydrogen, electrostatic, or van der waals interactions, however, binding may also be covalent. The ligand may be, e.g., a small organic molecule, a peptide, polypeptide, nucleic acid, polysaccharide, lipid, etc. Examples of ligands of the invention include, but are not restricted to, agonists and antagonists for cell membrane receptors such as G-protein coupled receptors; ion channels; toxins and venoms; viral epitopes; hormones (e.g. opiates, steroids, etc.); hormone receptors; transcription factors; tumor suppressors; growth factors and their receptors; chemokines and their receptors; peptides, enzymes; enzyme substrates; cofactors; drugs; lectins; sugars; oligosaccharides proteins and monoclonal antibodies. Such ligands can be obtained or isolated by methods well known in the art. Typically, the ligand is labeled either directly or indirectly, as described below.

A “compound” of interest is any molecule that competes with the ligand for binding to the target. The compound is also a potential ligand for the target, as described above. However, the compound may not have a known binding specificity for the target. The assays described herein are used to determine the binding specificity of the compound for the target.

The term “target” is a relative term that refers to a molecule that binds to or interacts with a ligand molecule. Typically the nature of the interaction or binding is non-covalent, e.g., by hydrogen, electrostatic, or van der waals interactions, however, binding may also be covalent. The target is typically a protein, but may be, e.g., a small organic molecule, a peptide, a polysaccharide, or a polynucleotide. The target, ligand, and potential ligand compound can be recombinant, derived from natural sources, or naturally occurring.

Whether naturally occurring, isolated from naturally occurring sources, recombinantly produced, or chemically produced, the amino acid sequences of polypeptide targets and ligands which may be used in the assays of the invention need not be identical to the reported sequence of the genes encoding them. Polypeptide targets, ligands, and potential ligands may comprise altered sequences in which amino acid residues are deleted, added, or substituted resulting in a functionally equivalent product.

Any number of methods routinely practiced in the art can be used to identify and isolate a target protein's binding site or active site. These methods include but are not limited to, e.g., mutagenesis of the gene encoding the target protein and screening for disruption of binding in a co-immunoprecipitation assay.

A tissue or biological sample of interest in which localized binding is to be investigated is also obtained. Any tissue, receptive to ligand binding whether cultured or not, can be used. Such samples include, but are not limited to, tissue isolated from humans. Biological samples may also include sections of tissues such as frozen sections taken for histologic purposes, e.g., biopsies, or tissue samples such as blood, sputum, etc., or cultured cells, e.g., explants, primary cultures, and transformed cells. A biological sample is typically obtained from a eukaryotic organism, preferably eukaryotes such as fungi, plants, insects, protozoa, birds, fish, reptiles, and preferably a mammal such as rat, mice, cow, dog, guinea pig, or rabbit, and most preferably a primate such as chimpanzees or humans. Particularly preferred are biopsy samples, autopsy, fresh cultured samples and tissues that have been preserved in paraffin or wax. These tissues can be rehydrated and tested in accordance with the assays herein. For example, tissues known or suspected of being in a disease state can be tested in comparison with those that are healthy. Ligands can be tested against various tissues to determine whether binding is or can be localized to any of them. The assays are typically conducted in situ where the protein receptor or target is present in place within its natural cell or tissue.

The term “recombinant” when used with reference, e.g., to a cell, or nucleic acid, or vector, indicates that the cell, or nucleic acid, or vector, has been modified by the introduction of a heterologous nucleic acid or the alteration of a native nucleic acid, or that the cell is derived from a cell so modified. Thus, for example, recombinant cells express genes that are not found within the native (non-recombinant) form of the cell or express native genes that are otherwise abnormally expressed, under expressed or not expressed at all. In the methods of the invention, first, the tissue of interest is incubated with a potential binding compound of interest for a time sufficient to permit binding of the ligand to a protein target if present in the tissue. Generally those of skill in the art know appropriate incubation times or can readily ascertain them by testing with tissue known to have the protein target.

In one embodiment of the assay of the invention, a ligand compound is obtained that may be specific for a target protein of interest. Then, the sample, e.g., tissue or cells of interest, is exposed to the second ligand which has a known binding specificity. The level of binding of the second ligand-protein binding complex is measured and the level is inversely proportional to the extent of the competitive binding by the first ligand of interest. Thus, if the level of binding of the second ligand is low this indicates that the level of binding of the first ligand of interest was high and was localized to the same target protein or peptide. It should be evident that these assays are most accurately run when done in conjunction with control binding of the second ligand to the tissue of interest in the absence of the first ligand.

The level of binding can be detected in any of the many ways known and available in the art. Typically the second ligand will be labeled either directly or indirectly in a manner so that its presence is readily indicated. Alternatively, an additional ligand that distinguishes between the first and second ligand, by binding the second, but not the first can be labeled and used to detect the level of binding (see, for example, U.S. Pat. No. 5,578,452 that describes a method for enhancing immunochemical staining). In one embodiment, the target protein from the in situ sample is solubilized and fixed to a membrane or substrate prior to binding and detection. In another embodiment, binding and detection are observed in the fixed tissue or cell sample.

This invention relies on routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

Competitive Assays

Surprisingly, the methods of the present invention permit the screening of ligand-binding interactions tissues or cells in situ. It is surprising that competitive inhibition could be detected in the complex milieu of a biological tissue or cell rather than in controlled solution with pure compounds. Further, it is surprising that this assay could be accomplished in an array so that multiple tissues or ligands could be tested at once. In situ methods are well known to those of skill in the art. See, e.g., Chapter 14 of Current Protocols, supra; and In Situ Hybridization Protocols (Cho, ed. 1994).

In competitive assays such as these, the binding of a compound (analyte) to a target protein present in the sample is measured indirectly by measuring the amount of an added (exogenous) second ligand that displaces (or competes away) the compound from the target. In one embodiment of a competitive assay, the target molecule in the sample is incubated with the first ligand compound of interest. Then, a known amount of a second ligand, e.g., an antibody that specifically binds to the target, is added to the sample. Unbound second ligand is removed from the sample. The amount of the second ligand bound to the target is proportional to the amount of displaced ligand compound. The amount of the second ligand, e.g., the antibody, bound to the target may be determined either by measuring the amount of second ligand present in the protein/ligand complex or, alternatively, by measuring the amount of remaining uncomplexed second ligand. The amount of bound or unbound may be detected by providing a detectable moiety.

Throughout the assays, incubation and/or washing steps may be required after each combination of reagents. Incubation steps can vary from about 5 seconds to several hours, preferably from about 5 minutes to about 24 hours. The incubation time will depend upon the assay format, analyte, volume of solution, concentrations, and the like. Usually, the assays will be carried out at ambient temperature, although they can be conducted over a range of temperatures, such as 11° C. to 40° C.

Detectable Moieties

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most labels useful in such methods can be applied to the present invention. Thus, a label is any composition detectable by spectroscopic, photochemical, biochemical, immunochemical, electrical, optical or chemical means. Useful labels in the present invention include magnetic beads (e.g., DynabeadsTM), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., CAT, LacZ, horse radish peroxidase, alkaline phosphatase, and others commonly used in an ELISA), and colorimetric labels such as colloidal gold or colored glass or plastic (e.g., polystyrene, polypropylene, latex, etc.) beads, substrates, cofactors, inhibitors, or fluorescent moieties (e.g., fluorescein and its derivatives, rhodamine, danysl and the like), chemiluminescent moieties (e.g., luciferin and 2,3-dihydrophthalazinediones), radioactive labels, magnetic particles, and the like. Examples of direct ligand binding and detection include the use of biotin labeled nucleotides or the use of digoxigenin, including photobiotinylation. These molecules can be used as the ligand binding component. They can be readily captured by their anti-ligand, e.g. avidin or streptavidin in the case of biotin and an anti-digoxigenin antibody, bound on a suitable substrate. Molecules which do not bind the anti-ligand can be collected and captured, by for example passing them through a streptavidin column. A wide variety of labels suitable for labeling and conjugation techniques for labeling are reported extensively in both the scientific and patent literature. The choice of label depends on the sensitivity required, ease of conjugation of the compound, stability requirements, available instrumentation and disposal provisions.

The label may be coupled directly or indirectly to the desired component of the assay according to methods well known in the art. As indicated above, a wide variety of labels may be used, with the choice of label depending on the sensitivity required, the ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Thyroxine, and cortisol can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol (for a review of various labeling or signal producing systems which may be used, see, U.S. Pat. No. 4,391,904).

Chemilumination is a method that is particularly preferred and is well adapted to the screening of multiple samples and multiple arrays. Chemiluminescent protocols include, but are not limited to, the ECL Western Blotting System (Amersham). (See, e.g., U.S. Pat. No. 4,745,077) or Lumigen PS-3 blotting applications (Lumigen, Inc.) that utilize enzymatic generation of acridinium esters. Reaction of the acridan substrate with an HRP label generates acridinium ester intermediates which react with peroxide at alkaline pH to produce high-intensity chemiluminescence. See, e.g., Akhavan-Tofti et al., Clin. Chem. 41(9), 1368-9 (1995).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by microscopy, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected directly by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need to be labeled and the presence of the target antibody is detected by simple visual inspection.

Antibodies

In one embodiment, the second ligand is an antibody that is known to bind to the target. In another embodiment, the labeling agent is a second antibody bearing a label, which binds to the target of interest but does not compete for binding with the ligand and/or compound. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see generally, Kronval, et al. J Immunol., 111:1401-1406 (1973); and Akerstrom, et al. J. Immunol., 135:2589-2542 (1985)).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The Nterminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of wellcharacterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993)). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The term “immunoassay” is an assay that uses an antibody to specifically bind an antigen. The immunoassay is characterized by the use of specific binding properties of a particular antibody to isolate, target, and/or quantify the antigen. Preferred immunoassays include, e.g., ELISA assays.

The phrase “specifically (or selectively) binds” to a target protein or antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised any particular polypeptide target, or portions thereof, can be selected to obtain only those polyclonal antibodies that are specifically immunoreactive with only the target polypeptide and not with other proteins. This selection may be achieved by subtracting out antibodies that cross-react with molecules such as other related polypeptides. For example, antibodies that specifically bind to polymorphic variants, alleles, orthologs, and conservatively modified variants but not, e.g., orthologs or the target can be selected. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective reaction will be at least twice background signal or noise and more typically more than 10 to 100 times background.

The labeled antibodies of the invention can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7^{th} ed. 1991).

Ligand Compounds

The ligand compounds tested as target binders in the present invention can be any small chemical compound, or a biological entity, such as a protein, sugar, nucleic acid, or lipid. Often, test compounds will be small chemical molecules and peptides. Essentially, however, any chemical compound can be used as a potential ligand in the assays of the invention. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g. U.S. Pat. No. 5,539,083), antibody libraries (see, e.g., Vaughn et al., Nature Biotechnology, 14(3):309-314 (1996) and PCT/US96/10287), carbohydrate libraries (see, e.g. Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

High Throughput Assays

The assays of the present invention offer the advantage that many in situ samples can be processed in a short period of time. High throughput assays for the presence, absence, or quantification (e.g., for binding specificity) of particular compound are well known to those of skill in the art. Thus, for example, U.S. Pat. No. 5,559,410 discloses high throughput screening methods for protein and U.S. Pat. Nos. 5,576,220 and 5,541,061 describe methods for ligand/antibody binding. In the subject invention, the assays can be surprisingly, but easily, adapted to an array. For example, tissue samples can be spotted on a membrane, such as a nitrocellulose membrane and then treated as required by the assay. This method has been conveniently applied to samples of tissues preserved or prepared in wax. Typically the wax samples are rehydrated in octane or xylene, washed with ethanol and then microwaved and cooled. They are then readily spotted onto a membrane and fixed on the membrane. See, PCT Application No. 98/18399 or U.S. Ser. No. 08/925,818, commonly assigned.

In one embodiment, the invention provides solid phase based in vitro or in situ assays in a high throughput format, where the cell or tissue expressing a target is attached to a solid phase substrate. In the high throughput assays of the invention, it is possible to screen up to several thousand different potential ligands in a single day. In particular, each well of a microtiter plate can be used to run a separate assay against a selected potential ligand, or, if concentration or incubation time effects are to be observed, every 5-10 wells can test a single ligand. Thus, a single standard microtiter plate can assay about 96 ligands. If 1536 well plates are used, then a single plate can easily assay from about 100- about 1500 different compounds. It is possible to assay many plates per day; assay screens for up to about 6,000, 20,000, 50,000, or 100,000 or more different compounds are possible using the integrated systems of the invention.

### EXAMPLES

### Example 1

Identification of Compound-protein Interactions for Use in a High Throughput Screening Assay

a. Antibody Synthesis.

Analyze the peptide sequence of the protein to be targeted and synthesize peptides that code for the binding site or active site of the protein. Make monoclonal or polyclonal antibodies by injecting mice or rabbits with the purified peptide. Affinity purify the antibody through a peptide column.

b. Protein Synthesis.

Express the protein to be targeted by cloning the gene into a suitable expression vector. Grow clones into a 96 or 384 well microtiter plate and perform crude extracts of protein, or purify the protein and seed into microtiter plates or membranes. Affix the protein to the bottom of the plate or membrane.

c. Compound-antibody Competition Assay.

Add the compounds to be tested to each well containing the expressed protein and incubate. Wash the compounds from the wells. Add antibody to each well and incubate. Wash antibody from the wells.

d. Detection of Binding.

Binding interactions between the antibody and the protein can be detected in a number of ways. The primary antibody can be directly labeled (i.e., with biotin, digoxigenin, fluorescein, radioisotope) or a labeled secondary antibody targeted toward the primary antibody can be used to detect binding of the primary antibody. The amount of binding can then be detected by existing plate reading instrumentation. The difference in signal between the antibody bound in the absence of compound (positive control signal) and in the presence of compound is proportional to the extent of competitive binding by the compound.

### Example 2

Detection of Compound-protein Binding in Cells or Tissues in Situ

a. Antibody Synthesis.

Analyze the peptide sequence of the protein to be targeted and synthesize peptides that code for the binding site or active site of the protein. Make monoclonal or polyclonal antibodies by injecting mice or rabbits with the purified peptide. Affinity purify the antibody through a peptide column.

b. Preparation of Tissue or Cells for Binding Assay.

Cells in culture or frozen sections of tissues can be used either directly, after permeabilization, or after fixation. With paraffin archival tissues, it is preferable to rehydrate the tissue slice using antigen retrieval methods (deparaffinization with xylene or octane, followed by heat treatment in water or buffered solution) or also see U.S. patent application Ser. No. 08/925,818, incorporated by reference herein.

c. Binding Compound to Tissue or Cells.

Incubate compound with tissue section, wash. Then incubate with primary antibody, wash.

d. Detection of Binding.

Binding interactions between the compounds and the protein within the cells or tissues can be detected in a number of ways. The primary antibody can be directly labeled (i.e., with biotin, digoxigenin, fluorescein, radioisotope) or a labeled secondary antibody targeted toward the primary antibody can be used to detect binding of the primary antibody. Binding can then be detected by direct microscopy or fluorescence microscopy. The positive signal generated by the primary antibody should decrease if compound is effectively binding to the active site of the protein.

### Example 3

Detection of Compound-protein Binding in Tissues on an ELISA Format by Solubilization of Protein Prior to Binding

a. Tissue Rehydration.

The paraffin sections are initially deparaffinized and rehydrated by treatment with successive incubations in octane or xylene for four treatments of 5 minutes each, followed by two successive washes each for one minute in 100% ethanol, 50% ethanol, and deionized water. The rehydrated sections are then microwaved for two five minute intervals in 0.01 moles/liter citrate buffer, pH 6.0 (i.e., Microwaving Buffer, BioTek Solutions, Inc., Product #MWB101). The samples are cooled for 20 minutes.

b. Solubilization and Spotting.

The rehydrated sections are then treated with 0.5% trypsin for one hour each at 37° C., and the solubilized tissue sample is then heated to 98 degrees C for 20 minutes and centrifuged. The soluble fraction of the sample is spotted (6 microliters) onto nitrocellulose membrane and fixed with 4% paraformaldehyde in phosphate buffered saline (PBS) for 30 minutes, followed by two washes with PBS.

c. Compound Incubation.

The membranes are then spotted with individual compound containing solutions and allowed to interact. The membranes are then washed.

d. Blocking.

The membrane is blocked by treatment with 5% nonfat dry milk (NFDM) for an hour at room temperature, and the blocking solution is then aspirated.

e. Reaction with Primary Antibody.

The membrane is incubated with 2 ml of solution containing the primary mouse anti-human peptide antibody in 0.5% NFDM in PBS for one hour at room temperature on a rotator, then washed four times with PBS.

f. Reaction with Secondary Antibody.

The membrane is incubated with 2 ml of goat anti-mouse IgG HRP (1:2000) in 0.5% NFDM in PBS on a rotator for one hour at room temperature, then the solution is aspirated and the membrane is washed four times in PBS. The signal is then detectable by a chemilumination assay (i.e., ECL Western blotting system, Amersham). The presence of successful binding by the compound is detected as a decrease in signal over the antibody binding in the absence of compound.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for in situ detection of target-ligand binding in a biological sample, the method comprising the steps of:
(a) incubating the in situ biological sample with a first potential ligand of interest; then
(b) contacting the biological sample with a second ligand having a known binding specificity to a target protein present in the biological sample to create a second ligand-protein complex;
(c) removing unbound second ligand from the second ligand-protein complex; and
(d) detecting the level of binding of the second ligand to the target protein, wherein the level of binding of the second ligand to the target protein is proportional to the extent of competitive binding by the first ligand.

2. The method of claim 1, wherein the biological sample is rehydrated tissue from a paraffin sample.

3. The method of claim 2, wherein the tissue is heated in a microwave after rehydration.

4. The method of claim 1, wherein the biological sample is tissue that is treated with trypsin and then heated to 98° C. prior to incubation.

5. The method of claim 1, wherein the biological sample is placed on a membrane prior to incubation.

6. The method of claim 1, wherein the biological sample is placed in an array of over 4 positions on a membrane.

7. The method of claim 1, wherein the second ligand is detected by chemoillumination.

8. The method of claim 1, wherein the second ligand is detected indirectly by a second antibody capable of binding to the second ligand.

9. The method of claim 1, wherein the second ligand is detected by fluorescence microscopy.

10. The method of claim 1, wherein the second ligand is labeled with a detectable moiety.

11. The method of claim 1, wherein the second ligand is an antibody.

12. The method of claim 11, wherein the antibody is labeled with a detectable moiety.

13. The method of claim 1, wherein the biological sample is contacted with a known amount of second ligand.

14. The method of claim 1, wherein the level of binding of the second ligand is detected by measuring the amount of second ligand-protein binding complex.

15. The method of claim 1, wherein the level of binding of the second ligand is detected by measuring the amount of unbound second ligand.

16. The method of claim 1, wherein the biological sample is tissue.

17. The method of claim 1, wherein the biological sample is cells.

18. The method of claim 17, wherein the biological sample is cultured cells.
